(19) United States
(12) Patent Application Publication
(10) Pub. No.: US 2024/0193762 A1
(43) Pub. Date: Jun. 13, 2024

(54) SELECTING ANALYSIS ALGORITHMS TO USE TO GENERATE MEDICAL FINDINGS FOR MEDICAL IMAGES

(71) Applicant: RADIOLOGY PARTNERS, INC., El Segundo, CA (US)

(72) Inventors: Bartosz BREWINSKI, Mississauga (CA); Mike ANDORFER, Hartland, WI (US); Kelly DENNEY, Hilliard, OH (US)

(21) Appl. No.: 18/065,582

(22) Filed: Dec. 13, 2022

(51) Int. Cl. G06T 7/00; G06V 10/764; G06V 10/774; G16H 50/20

(52) U.S. Cl. CPC G06T 7/0012; G06V 10/764; G06V 10/774; G16H 50/20; G06T 2207/20081

(57) ABSTRACT

Provided are computer program product, system, and method for selecting analysis algorithms to process data. Feedback scores are received for classifications produced by a plurality of analysis algorithms processing training data. Training sets are generated including inputs comprising attributes of the analysis algorithms, attributes of the training data, and the feedback scores. An orchestration supervisor, implementing a machine learning algorithm, is trained with the inputs from the training sets to produce the feedback scores. The trained orchestration supervisor is deployed to receive inputs comprising attributes of the analysis algorithms and attributes of received data to output algorithm scores for the analysis algorithms. The algorithm scores are used to select at least one analysis algorithm of the analysis algorithms. The received data is forwarded to the selected at least one analysis algorithm to generate at least one classification.

### BACKGROUND OF THE INVENTION

### 1. Field of the Invention

The present invention relates to a computer program product, system, and method for selecting analysis algorithms to use to generate medical findings for medical images.

### 2. Description of the Related Art

Medical imaging software analyzes and interprets digital medical images and produces diagnoses or medical findings of detected conditions in the medical images. The medical image may be in a format such as Digital Imaging and Communications in Medicine (DICOM) format, in which metadata is provided with the image. Medical image analysis software may use deep learning and machine learning algorithms to process the digital medical image and image metadata to extract features from the image and provide a diagnosis and classification of the extracted features, such as detection of a type of tumor, lesion, embolism, hemorrhage, nodule, etc., and classify the detected condition, e.g., normal, abnormal, as well as measure dimensions of the detected condition or feature. Medical image analysis software may also process hundreds of images at a time and flag images with findings that need further consideration to expedite radiologist processing by focusing their attention on images that may have a problematic detected condition.

A provider of medical image analysis software may charge a fee to analyze each medical image, on a per use basis or a subscription and license fee. Further, healthcare providers may prefer to use and consider a group of medical image analysis software that has received Federal Drug Administration (FDA) approval.

There is a need in the art to assist consumers of medical image analysis software to select available medical image analysis software programs to use to process medical images.

### SUMMARY

Provided are computer program product, system, and method for selecting analysis algorithms to process data. Feedback scores are received for classifications produced by a plurality of analysis algorithms processing training data. Training sets are generated including inputs comprising attributes of the analysis algorithms, attributes of the training data, and the feedback scores. An orchestration supervisor, implementing a machine learning algorithm, is trained with the inputs from the training sets to produce the feedback scores. The trained orchestration supervisor is deployed to receive inputs comprising attributes of the analysis algorithms and attributes of received data to output algorithm scores for the analysis algorithms. The algorithm scores are used to select at least one analysis algorithm of the analysis algorithms. The received data is forwarded to the selected at least one analysis algorithm to generate at least one classification.

In certain implementations, the training data comprises training medical images and the received data comprises a received patient medical image for a patient. The classifications produced by the analysis algorithms comprise training medical findings for the training medical images and patient medical findings for the received patient medical image. The feedback scores are determined by at least one radiologist reviewing the training medical findings produced for the training medical images.

### BRIEF DESCRIPTION OF THE DRAWINGS

FIG. 1 illustrates an embodiment of a computing environment of an orchestration server to select analysis algorithms to use to generate medical findings for patient medical images.

FIG. 2 illustrates an embodiment of a score vector for an analysis algorithm.

FIG. 3 illustrates an embodiment of a training set used to train an orchestration supervisor machine learning model.

FIG. 4 illustrates an embodiment of operations to train the orchestration supervisor.

FIG. 5 illustrates an embodiment of operations to select analysis algorithms to use to generate medical findings for a patient medical image.

FIG. 6 illustrates an embodiment of operations to rank analysis algorithms by aggregating score vectors for the analysis algorithms.

FIG. 7 illustrates an embodiment of operations to rank analysis algorithms by using a learning-to-rank machine learning model that processes the score vectors for the analysis algorithms.

FIG. 8 illustrates an embodiment of operations to retrain the orchestration supervisor for an updated analysis algorithm.

FIG. 9 illustrates a computing environment in which the components of FIG. 1 may be implemented

### DETAILED DESCRIPTION

There are numerous available image analysis software programs that health care providers and radiologists may select from to use to analyze patient medical images to generate medical findings on detected medical conditions and measurements of the condition. One problem health care providers face is how to select the image analysis software program most suitable given the context for the particular medical image being processed.

Described embodiments provide improvements to computer technology for selecting image analysis software to process a medical image to provide findings for the medical image by training an orchestration supervisor, implementing a machine learning algorithm, to select among available analysis algorithms, such as image analysis software. Training sets are generated for the analysis algorithms including inputs comprising attributes of the analysis algorithms, attributes of training medical images for which the training medical findings are generated, and feedback scores from a reviewing radiologist for the medical findings produced by the analysis algorithms. The orchestration supervisor is trained with the inputs in the training sets to produce the feedback scores for the medical findings produced by the inputs. This trained orchestration server may then be deployed to receive as inputs information on the analysis algorithms and received patient medical images to predict scores indicating an accuracy, user experience, performance, and worth of the medical findings that will be produced by the analysis algorithms. These scores predicting the utility of the analysis algorithms may then be used to order or rank the analysis program so the highest ranked, or best, analysis programs may be selected to generate medical findings for the received patient medical image.

In this way, the described embodiments provide improved technology for training and using an orchestration supervisor to optimize selection of the most suitable analysis algorithms to use to process a patient medical image to generate medical findings for the received patient medical image. For instance, the orchestration supervisor provides an automated method to dynamically select a best analysis algorithm (or set of analysis algorithms) to use given a set of medical input data. This allows an organization to deploy multiple algorithms that provide a similar type of analysis. The orchestration supervisor algorithm is trained to prioritize the highest scoring algorithms based on radiologist/physician feedback (and accuracy). This prioritization reduces time to deployment because the weakest/lowest scoring algorithms may be automatically bypassed and not used for the particular image analysis that is currently required.

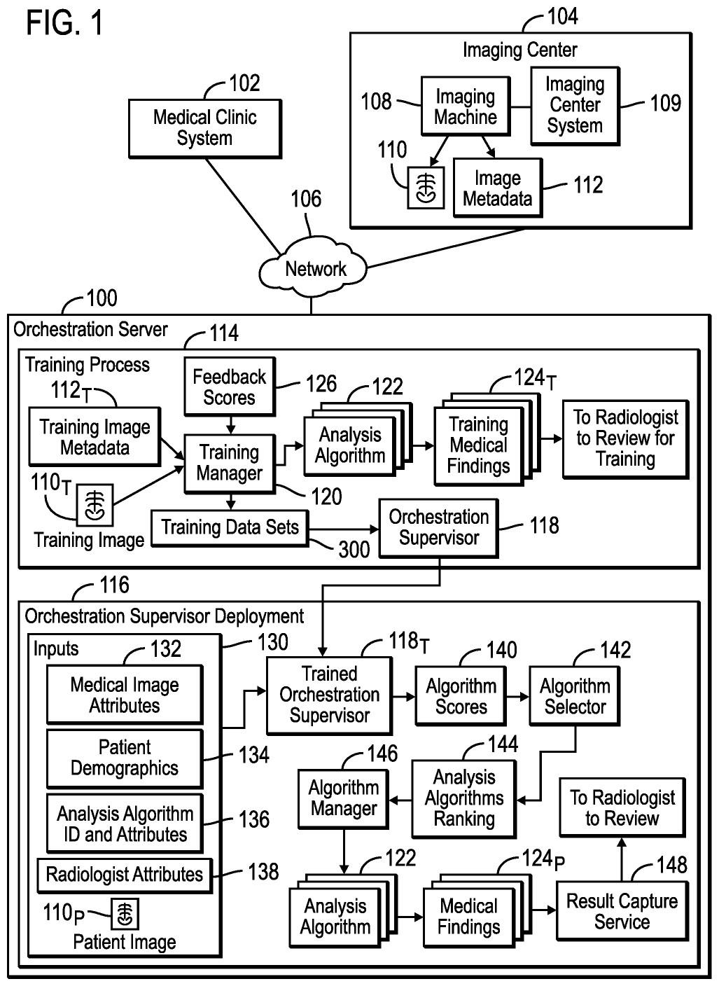

FIG. 1 illustrates an embodiment of a computing environment in which embodiments are implemented. An orchestration server 100 is in communication with a medical clinic system 102 and imaging center 104 over a network 106. The medical clinic 102 may comprise a radiology center or other type of medical clinic requiring imaging, such as a hospital, cardiology practice, oncology practice, orthopedic practice, etc. The imaging center 104 includes an imaging machine 108, such as CT (computed tomography), MRI (magnetic resonance imaging), ultrasound, X-ray, fluoroscopy, angiography, mammography, breast tomosynthesis, PET (positron emission tomography), SPECT (single-photon emission computed tomography), Endoscopy, microscopy, whole slide imaging, OCT (optical coherence tomography), etc. The imaging machine 108 performs an examination of a patient to produce a medical image 110_{T}, 110_{P}, such as a digital medical image, and image metadata 112. The image metadata 112 may comprise information on the imaging machine 108, information on a data format of the image metadata, resolution, format to store, exchange and transmit medical images, e.g., Digital Imaging and Communications in Medicine (DICOM), information on the medical clinic 102 that is requesting the image, and information on the imaging center 104 to processes the image 110_{T}, 110_{P}, etc. The imaging center 104 further includes an imaging center system 109 to transmit the medical image 110_{P} and image metadata 112 to the orchestration sever 100 to further process.

Although only one medical clinic 102 and imaging center 104 are shown, there may be multiple medical clinics 102 and imaging centers 104 that interact with one or more instances of the orchestration server 100. In certain situations, the medical clinic system 102 and imaging center 104 may be at the same facility.

A medical image 110_{T} and the image metadata 112 are sent to the orchestration server 100 to process as part of a training process 114 and an orchestration supervisor deployment 116. In the training process 114 to train the orchestration supervisor 118, a training manager 120 receives a training image 110_{T} and image metadata 1127 that are generated by the imaging machine 108 for purposes of training an orchestration supervisor 118. Alternatively, the training image 110_{T} and image metadata 1121 may be a previously generated image 110_{P} and image metadata 112_{P} generated for a patient radiology order that are then reused, with appropriate patient waivers, permission, and scrubbing of personal identifiable information (PII), to retrain the orchestration server 100. The training manager 120 forwards the training image 100_{T} and training image metadata 112_{T} to each of available analysis algorithms 122, such as medical image analysis software programs. Analysis algorithms 122, implemented as computer programs in software or hardware (e.g., applications specific integrated circuit (ASIC)), are designed to receive a same data set, such as a medical image and determine a same type of result, such as a medical finding of a detected condition and measurement of the condition. For instance, the analysis algorithms 122 may process the medical image 110_{T}, 110_{P} to generate medical findings 124_{T}, 124_{P}, such as a size of an observed condition on a patient, including as a size of an abdominal aortic aneurysms (AAA), observed features, such as size, shape, etc., of an incidental thyroid nodule, ovarian cyst, non-incidental thyroid nodule, enlarged thyroid, simple ovarian cyst, etc.

The analysis algorithms 122 may implement a machine learning model, rule-based system, decision tree, etc., and are designed to determine the same type of result independently of other analysis algorithms. The analysis algorithms 122 may implement a detection or measurement algorithm. Examples of analysis algorithms 122 include, but are not limited to: detect normal or abnormal results, such as a Chest X-Ray, Brain CT scan; pathology detection, such as to detect pulmonary embolism, breast lesion, intracranial hemorrhage, rib fracture; and lesion detection and measurement, such as to detect and measure lung modules and incidental thyroid modules.

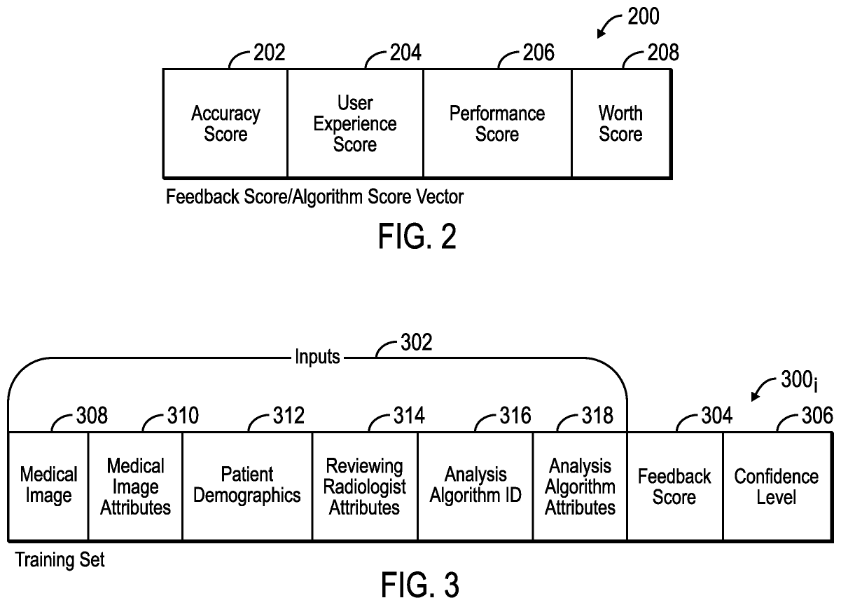

The training medical findings 124 are forwarded to a radiologist to review and provide feedback scores 126. In one embodiment, the feedback scores 126 may comprise a single value indicating a degree of utility of the medical finding. In an alternative embodiment, the feedback scores 126 from the radiologist may comprise score vectors 200, where a score vector 200, as shown in FIG. 2, for a medical finding and analysis algorithm pair is a multi-dimensional value indicating an accuracy score 202 providing a radiologists determination of an accuracy of the medical finding 124_{T} as of the condition shown in the training medical image 110_{T}; a user experience score 204 indicating a radiologist rating of the user experience interacting and viewing the medical findings 124_{T}; a performance score 206 indicating a performance of the algorithm 122, such as time it took the algorithm 122, e.g., if the algorithm takes two hours to run and the radiologist has long since reviewed the image 110_{T}, then the performance score is very low; and a worth score 208 indicating a value of the cost versus results, e.g., is it too expensive, a reasonable cost, or inexpensive given the output provided.

The accuracy score 202 may be automatically determined. For instance, if the algorithm 122 indicates it has found something, but a radiologist says the image 110_{T} does not have the algorithm 122 finding, then the accuracy score 202 may indicate inaccurate, or low accuracy, by assuming the algorithm 122 is wrong due to the discrepancy with the radiologist.

The user experience score 204 may indicate the ease with which the radiologist interacts with the rendered results. For instance, some algorithm 122 vendors provide an application that must be installed on the radiologist's workstation for viewing the results. Other algorithms 122 may allow the results to be viewed in the radiologist's native Picture Archiving and Communications System (PACS), but the results may be presented poorly (hard to read/interact with) in the PACS. Even though the accuracy of what is presented in the native PACS is good, the user experience score 204 may still be low if the radiologists have difficulty viewing and interpreting the results in the native PACS.

The training manager 120 receives the radiologist feedback scores 126, and from those generates training datasets 300. FIG. 3 illustrates an embodiment of a training dataset 300; instance generated for one medical finding/feedback score/analysis algorithm tuple. A training set 300; includes inputs 302 to the orchestration supervisor 118 machine learning model to generate as output the feedback score 304 from the radiologist and a confidence level 306 at which the orchestration supervisor 118 is trained to produce the feedback score 304. The confidence level 306 indicates a degree of confidence, such as a percentage, in the prediction of the feedback score 304 from the inputs 302. The confidence level 306 may be set to a relatively high value such as 80% or more to train the orchestration supervisor 118 to produce the feedback score 304 from the inputs 302 with a high probability or confidence level 306. A confidence level, also known as a confidence interval or confidence score, may comprise a number between 0 and 1, or other numerical range or fixed number of levels (e.g., high, medium or low), that represents the likelihood that the output of the orchestration supervisor 118 machine learning model correctly predicts the feedback score 304 based on the inputs 302. The confidence level 306 comprises a margin of error in the cost function of the feedback score 304 and the output produced by the trained orchestrations supervisor 118, such that if the confidence level is a high confidence level, e.g., 95% or more percent, then the orchestration supervisor 118 is trained to produce the feedback score 304 with a margin of error within 5%. In alternative embodiments, a high confidence level at which to train the orchestration supervisor 118 to produce the feedback scores 304 may comprise other confidence levels at which the user believes appropriate to train the orchestration supervisor 118 to produce goal results.

The inputs 302 may include medical image attributes 310, such as information on the imaging machine 108 used to capture the medical image 110_{T}, 110_{P}, data format of image 110_{T}, 110_{P}, e.g., DICOM, image resolution, type of procedure, procedure code, and procedure order, e.g., referring physician, reason for exam, location of the patient, location of the medical clinic at which the patient is treated, location of the imaging center 104, etc. Additional information may include information about how the image was acquired, radiation doses, examination focus, etc.; patient demographics 312, such as age, gender, occupation, race, medical conditions, prescribed medication, location of patient, etc. (for instance, certain analysis algorithms may perform better in certain regions due to demographics in the region); reviewing radiologist attributes 314, such as radiology group, name, etc.; analysis algorithm identifier (ID) 316 of the analysis algorithm used to produce the training medical finding 124 for which the feedback score 126, 200 was provided; and analysis algorithm attributes 318, such as specific type of algorithm, e.g., machine learning, rules-based; category of results, such as detect abnormal/normal, pathology detection and type of pathology detection (e.g., pulmonary embolism, breast lesion, intracranial hemorrhage, rib fracture), lesion detection and measurement (e.g., lung nodules measurement tracking, incidental thyroid nodule detection and measurement), etc.; cost of running the algorithm 316 to generate a medical finding 124, time to receive results, etc.

The training manager 120 trains the orchestration supervisor 118 with the inputs 302 from the training data sets 300 to produce the feedback score 304 within the specified confidence level 306. Once the orchestration server 118 is trained, it is deployed as a trained orchestration supervisor 128 as part of an orchestration supervisor deployment 116. The orchestration supervisor deployment 116 may comprise a cloud based service, such as Software as a Service (Saas), that is offered to radiology groups or imaging facilities, hospitals, and other health care providers, to select analysis algorithms 122 to process a received patient medical image 112_{P} and generate patient medical findings 124_{P} and produce a radiology report. Alternatively, the orchestration supervisor deployment 116 may be located on a computer system at the medical clinic system 102 or imaging center 104.

Upon receiving from an imaging center 104 a patient image 110_{P}, the trained orchestration supervisor 128T receives as inputs 130 medical image attributes 132, patient demographics 134, analysis algorithm attributes ID and attributes 136, and radiologist attributes 138 of a radiologist to review the medical findings 124_{P} from the analysis algorithms 122. The inputs 132, 134, 136, and 138 may comprise the same type of inputs 310, 312, 314, 316, and 318, as described with respect to FIG. 3, used to train the orchestration supervisor 118. The trained orchestration supervisor 118_{T} processes the inputs 130 to produce algorithm scores 140 for the analysis algorithms 122, which may comprise a score vector 200 for each analysis algorithm 122. An algorithm selector 142 processes the algorithm scores 140 to rank the analysis algorithms 122 in a ranking 144.

In an embodiment where the algorithm scores 140 comprise one-dimensional scores, such as an accuracy score, the algorithm selector 142 may rank the analysis algorithms 122 by direct scores 140. In an embodiment where the algorithm scores 140 comprise multi-dimensional scores 200, the algorithm selector 142 may aggregate the score vectors 200 into a single aggregated score to use to rank the analysis algorithms. Alternatively, the algorithm selector 142 may comprise machined-learned ranking (MLR) or learning-to-rank model to process the score vectors 200 to determine a ranking of the analysis algorithms 122. Further, the algorithm selector 142 may implement a rules-based, decision tree or regression analysis algorithm to determine a ranking 144 of the analysis algorithms 122. An algorithm manager 146 may then use the ranking 144 to select a predetermined number of analysis algorithms 122 to which to provide the patient image 110_{P} and any image metadata 112_{P} to the selected analysis algorithms 122 to produce independent patient medical findings 124_{P} from the patient medical image 110_{P}. The selected number of analysis algorithms 122 may alternatively be determined by selecting analysis algorithms whose combined cost is within a budget. The patient medical findings 124_{P} are provided to a result capture service 148 to forward to the medical clinic system 102 for radiologist review and use of the one or more medical findings 124_{P} in providing radiology services.

In the described embodiments, the analysis algorithm 122 may be located at the orchestration server 100. Alternatively, the analysis algorithms 122 may comprise cloud-based services and the medical images 110_{T}, 110_{P} are sent to analysis algorithms at 122 different network locations in the cloud to process.

In certain embodiments, various of the programs, such as the orchestration supervisor 118, analysis algorithms 122, and algorithm selector 142 may use machine learning and deep learning algorithms, such as decision tree learning, association rule learning, neural network, inductive programming logic, support vector machines, Bayesian network, recurrent neural networks (RNN), Feedforward neural networks, Convolutional Neural Networks (CNN), deep convolutional neural networks (DCNNs), Generative Adversarial Network (GAN), etc. For artificial neural network program implementations, the neural network may be trained using backward propagation to adjust weights and biases at nodes in a hidden layer to produce a classification, such as algorithm scores 140, 200, a medical finding 124_{T}, 124_{P}, and ranking 144. In backward propagation used to train a neural network machine learning module, biases at nodes in the hidden layer are adjusted accordingly to produce the algorithm scores 140, 200, medical finding 124_{T}, 124_{P}, and ranking 144 based on the inputs, such as inputs 130, 302. Backward propagation may comprise an algorithm for supervised learning of artificial neural networks using gradient descent. Given an artificial neural network and an error function, the method may use gradient descent to find the parameters (coefficients) for the nodes in a neural network or function that minimizes a cost function measuring the difference or error between actual and predicted values for different parameters. The parameters are continually adjusted during gradient descent to minimize the error.

In backward propagation used to train a neural network machine learning module, such as the orchestration supervisor 118, 118_{T}, analysis algorithms 122, and algorithm selector 142, margin of errors are determined based on a difference of the calculated predictions and user rankings of the output. Biases (parameters) at nodes in the hidden layer are adjusted accordingly to minimize the margin of error of the error function. The algorithm selector 142 may be implemented using learning-to-rank and machine-learned ranking (MLR) models.

In an alternative embodiment, the orchestration supervisor 118, 118_{T}, analysis algorithms 122, and algorithm selector 142 may be implemented not as a machine learning module, but implemented using a rules based system to determine the medical findings 124_{P} from the inputs 302. The program components 118, 118_{T}, 122, and 142 may further be implemented using an unsupervised machine learning module, or machine learning implemented in methods other than neural networks, such as multivariable linear regression models.

The arrows shown in FIG. 1 between the components and objects in a memory of the orchestration server 100 represent a data flow between the components.

The network 106 may comprise one or more networks including Local Area Networks (LAN), Storage Area Networks (SAN), Wide Area Network (WAN), peer-to-peer network, wireless network, the Internet, etc.

Generally, program modules, such as the program components 120, 122, 118, 118_{T}, 142, 146, 148 may comprise routines, programs, objects, components, logic, data structures, and so on that perform particular tasks or implement particular abstract data types. The program components and hardware devices of the systems 100, 102, 104 of FIG. 1 may be implemented in one or more computer systems, where if they are implemented in multiple computer systems, then the computer systems may communicate over a network.

The program components 120, 122, 118, 118, 142, 146, 148 may comprise computer programs accessed by a processor from memory to execute. Alternatively, some or all of the program components 120, 122, 118, 118, 142, 146, 148, or computational portions thereof, may be implemented in separate hardware devices, such as Application Specific Integrated Circuit (ASIC) hardware devices.

The functions described as performed by the programs 120, 122, 118, 118, 142, 146, 148 be implemented as program code in fewer program modules than shown or implemented as program code throughout a greater number of program modules than shown.

Some or all of the components shown as implemented in the server 100, such as the programs 120, 122, 118, 118_{T}. 142, 146, 148, may be implemented in the imaging center system 109 or medical clinic system 102. In certain embodiments, the orchestration server 100 may comprise a cloud server providing cloud services for the training process 114 and orchestration supervisor deployment 116. The orchestration server 100 may also provide medical findings 124_{P}, and algorithm scores 140 to systems at different medical facilities, locations, hospitals, etc., to provide cloud-based services.

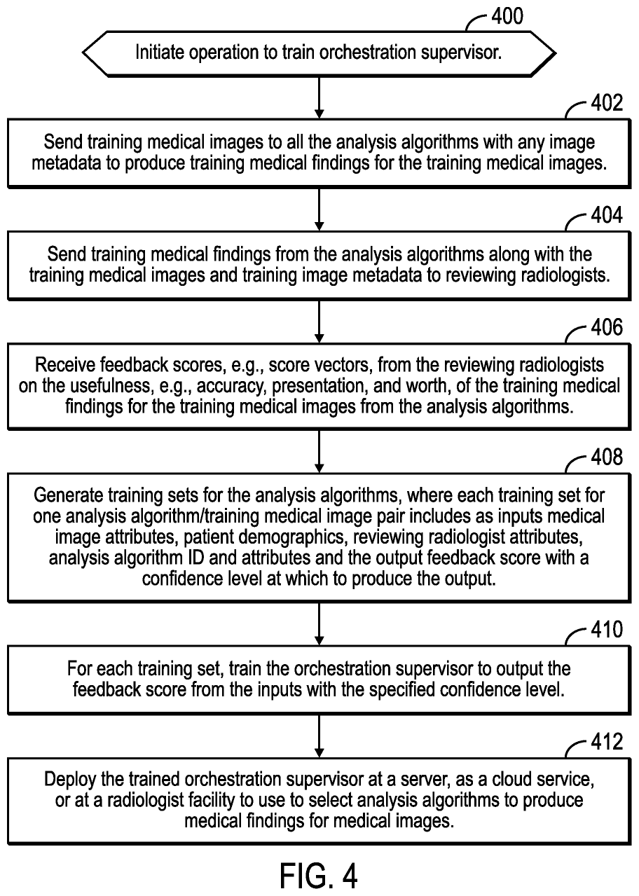

FIG. 4 illustrates an embodiment of operations performed by the training manager 120 to train the orchestration supervisor 118 to output accurate scores, such as score vectors 200, for a training medical image 110_{T}. Upon initiating (at block 400) an operation to train the orchestration supervisor 118, the training manager 120 sends (at block 402) training medical images 110_{T} to the analysis algorithms 122 with any image metadata 112_{T}, from the imaging center 104 when the training medical images 110_{T} were captured, to produce training medical findings 124_{T} for the training medical images 110_{T}. The training manager 120 sends (at block 404) training medical findings 124_{T} from the analysis algorithms 122 along with the training medical images 110 and training image metadata 112_{T} to radiologists to review to provide feedback on the utility of the training medical findings 124_{T}, e.g., accuracy 202, user experience 204, performance 206, worth score 208. The training manager 120 receives (at block 406) feedback scores 126, e.g., score vectors 200, from the radiologists on the usefulness, e.g., accuracy 202, user experience 204, performance 206, and worth or cost value, of the training medical findings from the analysis algorithms 122.

The training manager 120 generates (at block 408) training sets 300 for the analysis algorithms 122. Each training set 300 for one analysis algorithm 122/training medical image 110_{T} pair includes as inputs 302 the medical image 308, 110_{T} from which the medical finding 124T was generated, medical image attributes 310, patient demographics 312, reviewing radiologist attributes 314, the analysis algorithm ID 316 that generated the medical findings 1247 for which the feedback score 304 was provided, the analysis algorithm attributes 318, and the output feedback score 304 with a confidence level 306 at which to produce the output. For each generated training set 300, the orchestration supervisor 118 is trained (at block 410) to output the feedback score 304 from the inputs 302 with the specified confidence level 306, or margin of error. The training manager 120 may train the orchestration supervisor 118 by forming an input set, such as a matrix, of the inputs 302 for each of the training medical images 110_{T} having a number of columns equal to the number of input components 302 and a number of rows equal to a number of the training medical images 110_{T} and form an output set of the feedback scores 126 as a vector having the same number of rows of the input matrix. The trained orchestration supervisor 118_{T} is deployed (at block 412) at an orchestration server 100, such as a cloud service, an imaging center 104 or at a medical clinic system 102, to use to select a subset of the analysis algorithms 122, based on how the orchestration supervisor 118_{T} is trained, to produce medical findings 124_{P} for patient medical images 110_{P}. If the orchestration server 100 is operated as a cloud service, then an end user, such as radiologist or imaging center, may pay for use of deployed orchestration supervisor 118_{T}.

With the embodiment of FIG. 4, an orchestration supervisor 118 is trained to output feedback scores 126 from radiologists for different analysis algorithms 122 and training medical images 110_{T} based on inputs 302 concerning the medical image 110_{T}, analysis algorithms 122, and radiologist practice to receive the medical findings 124_{P} from the analysis algorithms 122. Once trained to produce the feedback scores 126 or score vectors 200 for the training medical findings 124_{T}, the trained orchestration supervisor 118_{T} may then be deployed to determine algorithm scores 140, e.g., 300, for the analysis algorithms 122 indicative of how they would perform given inputs 130 during real time operations. This estimated score is then used to rank the algorithms 122 to determine which algorithms 122 to run to process a patient image 110_{P} to determine medical findings 124_{P}. In this way, a subset of the algorithms 122 are selected to use that are most appropriate for the diagnosis for which the patient medical image 110_{P} is generated, for the type of imaging machine 108 used to produce the patient medical image 110_{P}, the preferences of the radiologist to receive the medical findings 124_{P}, location of the medical clinic, location of imaging center, location of patient, and the type of medical condition for which the patient medical image 110_{P} was ordered, as well as any other features of the analysis algorithm 122, imaging center 104, patient, and radiologist that are highly predictive of determining the utility of the medical finding produced by the analysis algorithm 122.

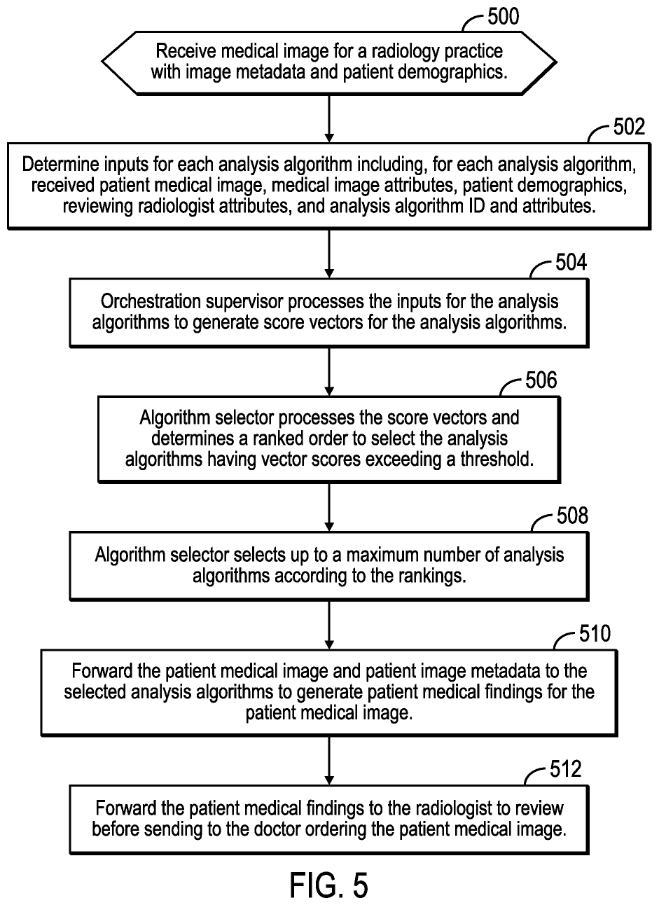

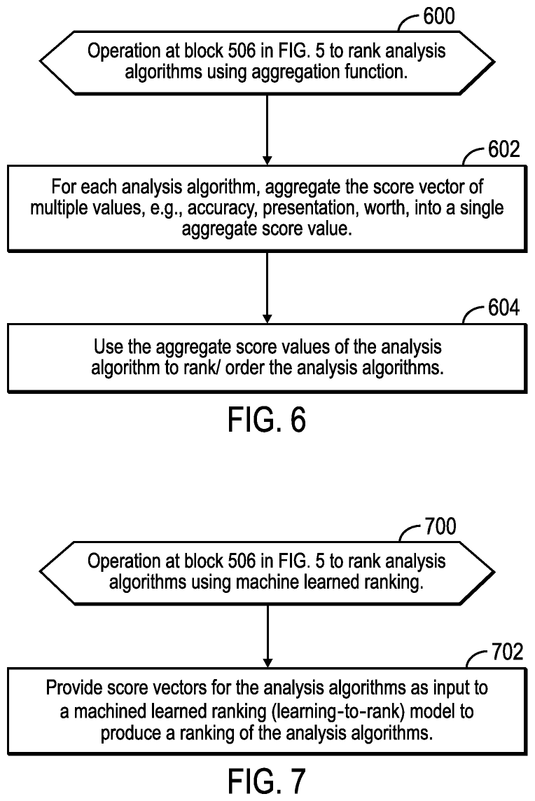

FIG. 5 illustrates an embodiment of operations for the trained orchestration supervisor 118_{T}, algorithm selector 142, algorithm manager 146, and result capture service 148 rank analysis algorithms 122 to select analysis algorithms 122 to use to generate patient medical findings 124_{P}. Upon receiving (at block 500) a patient medical image 110_{P} from an imaging center 104 for a medical clinic 102, the trained orchestration supervisor 118_{T} determines (at block 502) inputs 130 for each analysis algorithm 122 including the received patient medical image 110_{P}, medical image attributes 132, patient demographics 134, analysis algorithm ID and attributes 136, and reviewing radiologist attributes 138. The orchestration supervisor 118_{T} processes (at block 504) the inputs 130 for the analysis algorithms 122 to generate estimated algorithm scores 140, such as score vectors 200 or a single score, for each of the analysis algorithms 122. The algorithm selector 142 processes (at block 506) the estimated score vectors 200 and determines a ranking to select the analysis algorithms 122 having estimated score vectors 200 exceeding a threshold. A score vector 200 may exceed a threshold when each of its component values 202, 204, 206, and 208 exceed specific component thresholds. The algorithm selector 142 selects (at block 508) up to a maximum number of analysis algorithms 122 according to the rankings 144. The algorithm manager 146 forwards (at block 510) the patient medical image 110_{P} and patient image metadata 112_{P} to the selected analysis algorithms 122 to generate patient medical findings 124_{P} for the patient medical image 110_{P}. A result capture service 148 forwards (at block 512) the patient medical findings 124_{P} from the selected analysis algorithms 122 to the medical clinic system 102 for the radiologist to review and include in a radiology report before sending to the doctor ordering the patient medical image 110_{P}.

In one embodiment, if the estimated score comprises a single component value, and not a vector 200 having a multi-dimensional score, then the algorithm selector 142 may rank the analysis algorithms 122 according to the estimated single score value. In an alternative embodiment, the algorithm selector 142 may utilize a rules-based system, such as a decision tree, to process the score vectors 200 to determine the ranking order 144.

Once the selected analysis algorithms 122 are running processing the image 110_{P}, the radiologists may start reviewing the image 110_{P}. However, if a highly ranked analysis algorithm 122 indicates there is a medical finding 124_{P} in an image 110_{P}, then the radiologist may prioritize reviewing that medical image 110_{P} with the finding 124_{P} for reading in order to provide results to the patient as soon as possible. In this way, the medical findings 124_{P} from the analysis algorithms 122 assist the radiologist in prioritizing an image to review.

FIG. 6 illustrates an embodiment where the algorithm selector 142 uses an aggregation function to process the score vectors 200 for the analysis algorithms 122 to rank the analysis algorithms 122 using an aggregation function. Upon initiating (at block 600) the operation in block 506 in FIG. 5 to determine a ranking 144 of analysis algorithms 122, for each analysis algorithm 122, the algorithm selector 142 aggregates (at block 602) the score vectors 200 of multiple score components, e.g., accuracy 202, user experience 204, performance 206, worth 208, into single aggregate score values, one for each analysis algorithm 122. The algorithm selector 142 may apply a function to the score vectors 200 to convert the score components 202, 204, 206, 208 into a single value, such as by weighting and normalizing the values 202, 204, 206, 208 or through some other aggregation method. The aggregate score values are used (at block 604) to determine the ranking 144 of the analysis, such as order/rank according to the highest aggregate score values.

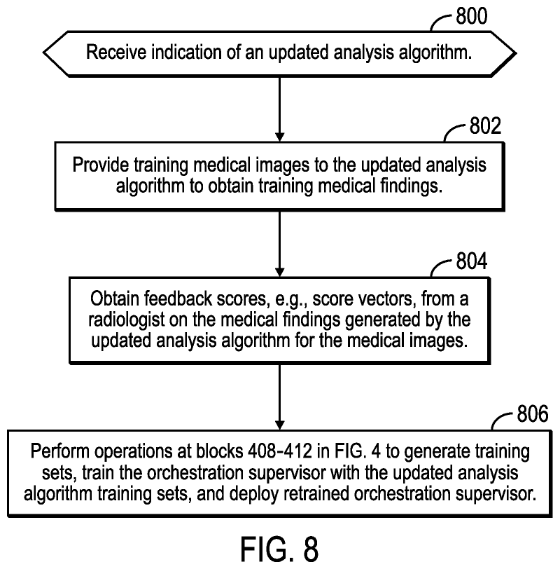

FIG. 7 illustrates an embodiment where the algorithm selector 142 implements a machine-learned ranking or learning-to-rank machine learning model to receive as input the score vectors 200 for all the analysis algorithms 122 and then produce a ranking. The algorithm selector 142 learning-to-rank machine learning model would be trained to output rankings based on user feedback of different input score vector 200 variations. Upon initiating (at block 600) the operation in block 506 in FIG. 5 to determine a ranking 144 of analysis algorithms 122, score vectors 200 for the analysis algorithms are provide (at block 602) as input to the algorithm selector 142, comprising machined learned ranking (learning-to-rank) model, to produce a ranking 144 of the analysis algorithms 122.

FIG. 8 illustrates an embodiment of operations performed by the training manager 120, or other component, to retrain the orchestration supervisor 118 when one of the analysis algorithms 122 is updated. Upon receiving (at block 800) indication of an updated analysis algorithm 122_{U}, training medical images 110_{T} are provided (at block 802) to the updated analysis algorithm 122_{U} to obtain training medical findings 124_{T} from the updated analysis algorithm 122_{U}. The training medical findings 124_{T} and training medical images 110_{T} are provided to a radiologist to obtain (at block 804) feedback scores 126, e.g., score vectors 200, indicating accuracy, user experience and/or performance, or other attribute scores, for the medical findings 124_{T} from the updated analysis algorithm 122_{U}. The training manager 120 may then perform (at block 806) operations at blocks 408, 410, and 412 in FIG. 4 to generate training sets 300, train the orchestration supervisor 118 with the updated analysis algorithm 122_{U} training sets 300, and then deploy the retrained orchestration supervisor 118_{T}.

With the embodiment of FIG. 8, the orchestration supervisor is retrained based on feedback of an updated analysis algorithm 122 to provide better predictions of a score for the updated analysis algorithm for a medical image that are used to rank the updated analysis algorithm with respect to other analysis algorithms to select based on the ranking.

In described embodiments, the training process utilizes training medical findings 124T separate from the patient medical findings 124_{P} produced in a deployed environment. In a further embodiment, the training medical findings 124_{T} used to produce training sets 300 to train the orchestration supervisor 118 may further include the patient medical findings 124_{P} along with feedback scores 126 from radiologists reviewing the patient medical findings 124_{P}. These training sets 300 based on real-time patient medical findings include as inputs 302 the inputs 130 used to produce the patient medical findings 124_{P} and the feedback score 304 based on the radiologist review of the patient medical findings 124_{P}. In this embodiment, real-time patient medical findings 124_{P} are also used to continually retrain the orchestration supervisor 118_{T}. The orchestration supervisor 118 may be trained during designated training periods or in real time while the orchestration supervisor 118 is deployed to determine patient medical findings 124_{P}.

In the described embodiments, the orchestration supervisor orders and selects analysis algorithms for the purpose of processing medical images to produce medical findings. In alternative embodiments, the orchestration supervisor and analysis algorithms may process data unrelated to medical images to produce output unrelated to medical findings in alternative operating environments. Examples of alternative operating environments may include selecting analysis algorithms for facial recognition, autonomous vehicle operation, natural language processing, character recognition, object recognition, etc. In such case the operations in FIGS. 4-8 may concern data sets to process other than medical images, and analysis algorithms to process the data sets to provide an output or finding/result for the data sets unrelated to analysis of medical images.

In such alternative embodiments, feedback scores are received for classifications produced by a plurality of analysis algorithms processing training data in a particular domain, not limited to analysis of radiology images. Training sets are generated for the analysis algorithms including inputs comprising attributes of the analysis algorithms, attributes of the training data for which the classifications are generated, and the feedback scores for the classifications. An orchestration supervisor implementing a machine learning algorithm, is trained to process the inputs from the training sets for the analysis algorithms to output the feedback scores for the analysis algorithms based on the inputs with a high confidence level. The trained orchestration supervisor is deployed in an operating environment to receive inputs comprising a data set to analyze, attributes of the analysis algorithms and attributes of the received data sets to output scores indicating a performance or utility of the analysis algorithms. The output scores are used to select at least one analysis algorithm of the analysis algorithms to process the received data set and generate a classification or finding for the received data set. The received data set is forwarded to the selected at least one analysis algorithm to generate at least one classification for the received set.

The present invention may be a system, a method, and/or a computer program product. The computer program product may include a computer readable storage medium (or media) having computer readable program instructions thereon for causing a processor to carry out aspects of the present invention.

The computer program product comprises a computer readable storage medium implemented using standard programming and/or engineering techniques to produce software, firmware, hardware, or any combination thereof. The described operations may be implemented as code or logic maintained in a “computer readable storage medium”. The term “code” and “program code” as used herein refers to software program code, hardware logic, firmware, microcode, etc. The computer readable storage medium, as that term is used herein, includes a tangible element, including at least one of electronic circuitry, storage materials, a casing, a housing, a coating, hardware, and other suitable materials. A computer readable storage medium may comprise, but is not limited to, a magnetic storage medium (e.g., hard disk drives, floppy disks, tape, etc.), optical storage (CD-ROMs, DVDs, optical disks, etc.), volatile and non-volatile memory devices (e.g., EEPROMs, ROMs, PROMs, RAMS, DRAMs, SRAMs, Flash Memory, firmware, programmable logic, etc.), Solid State Devices (SSD), computer encoded and readable punch cards, etc. The computer readable storage medium may further comprise a hardware device implementing firmware, microcode, etc., such as in an integrated circuit chip, a programmable logic device, a Programmable Gate Array (PGA), field-programmable gate array (FPGA), Application Specific Integrated Circuit (ASIC), etc. A computer readable storage medium, as that term is used herein, is not comprised solely of transmission signals and includes physical and tangible components. Those skilled in the art will recognize that many modifications may be made to this configuration without departing from the scope of the present invention, and that the article of manufacture may comprise suitable information bearing medium known in the art.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

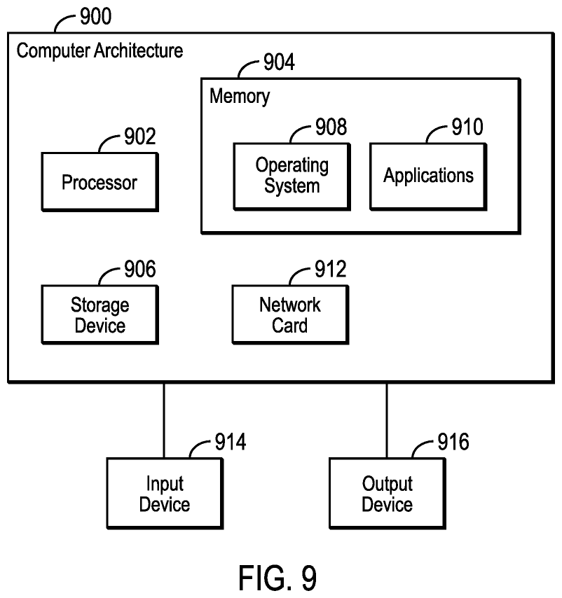

The computational components of FIG. 1, including the orchestration server 100, medical clinic system 102, and imaging center system 109 may be implemented in one or more computer systems, having a computer architecture as shown in FIG. 9, including a processor 902 (e.g., one or more microprocessors and cores), a memory 904 (e.g., a volatile memory device), and storage 906 (e.g., a non-volatile storage, such as magnetic disk drives, solid state devices (SSDs), optical disk drives, a tape drive, etc.). The storage 906 may comprise an internal storage device or an attached or network accessible storage. Programs, including an operating system 908 and applications 910 stored in the storage 906 are loaded into the memory 904 and executed by the processor 902. The applications 910 may include the training manager 120, orchestration supervisor 118, 118_{T}, algorithm selector 142, analysis algorithms 122, algorithm manager 146, result capture service 148, and any other programs.

The architecture 900 further includes a network card 912 to enable communication with the network 106. An input device 914 is used to provide user input to the processor 902, and may include a keyboard, mouse, pen-stylus, microphone, touch sensitive display screen, or any other activation or input mechanism known in the art. An output device 916, such as a display monitor, printer, storage, etc., is capable of rendering information transmitted from a graphics card or other component. The output device 916 may render the GUIs described with respect to figures and the input device 914 may be used to interact with the graphical controls and elements in the GUIs described above. The architecture 900 may be implemented in any number of computing devices, such as a server, mainframe, desktop computer, laptop computer, hand held computer, tablet computer, personal digital assistant (PDA), smartphone, wearable computer, telephony device, cell phone, etc.

The terms “an embodiment”, “embodiment”, “embodiments”, “the embodiment”, “the embodiments”, “one or more embodiments”, “some embodiments”, and “one embodiment” mean “one or more (but not all) embodiments of the present invention(s)” unless expressly specified otherwise.

The terms “including”, “comprising”, “having” and variations thereof mean “including but not limited to”, unless expressly specified otherwise.

The enumerated listing of items does not imply that any or all of the items are mutually exclusive, unless expressly specified otherwise.

The terms “a”, “an” and “the” mean “one or more”, unless expressly specified otherwise.

Devices that are in communication with each other need not be in continuous communication with each other, unless expressly specified otherwise. In addition, devices that are in communication with each other may communicate directly or indirectly through one or more intermediaries.

A description of an embodiment with several components in communication with each other does not imply that all such components are required. On the contrary a variety of optional components are described to illustrate the wide variety of possible embodiments of the present invention.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article or a different number of devices/articles may be used instead of the shown number of devices or programs. The functionality and/or the features of a device may be alternatively embodied by one or more other devices which are not explicitly described as having such functionality/features. Thus, other embodiments of the present invention need not include the device itself.

The foregoing description of various embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto. The above specification, examples and data provide a complete description of the manufacture and use of the composition of the invention. Since many embodiments of the invention can be made without departing from the spirit and scope of the invention, the invention resides in the claims herein after appended.

What is claimed is:

1. A computer program product for selecting one of a plurality of analysis algorithms to process a data set, the computer program product comprising a computer readable storage medium having computer readable program code embodied therein that is executable to perform operations, the operations comprising:
receiving feedback scores for classifications produced by a plurality of analysis algorithms processing training data;
generating training sets including inputs comprising attributes of the analysis algorithms, attributes of the training data, and the feedback scores;
training an orchestration supervisor, implementing a machine learning algorithm, with the inputs from the training sets to produce the feedback scores;
deploying the trained orchestration supervisor to receive inputs comprising attributes of the analysis algorithms and attributes of received data to output algorithm scores for the analysis algorithms;
using the algorithm scores to select at least one analysis algorithm of the analysis algorithms; and
forwarding the received data to the selected at least one analysis algorithm to generate at least one classification.

2. The computer program product of claim 1, wherein the training data comprises training medical images and the received data comprises a received patient medical image for a patient, wherein the classifications produced by the analysis algorithms comprise training medical findings for the training medical images and patient medical findings for the received patient medical image, and wherein the feedback scores are determined by at least one radiologist reviewing the training medical findings produced for the training medical images.

3. The computer program product of claim 2, wherein the inputs for the training sets include at least one attribute of the training medical images selected from the group consisting of a medical imaging machine used to create a training medical image, a medical condition to be detected by the analysis algorithm, information on an imaging center operating the medical imaging machine to produce the training medical image, and information on a medical clinic ordering the training medical image.

4. The computer program product of claim 2, wherein the inputs for the training sets include demographics of patients from which the training medical images were generated.

5. The computer program product of claim 2, wherein the inputs for the training sets include at least one attribute of the analysis algorithms selected from the group consisting of an identifier of the analysis algorithm for which the feedback score is provided, a type of condition detected and measured by the analysis algorithm, and a cost of running the analysis algorithm.

6. The computer program product of claim 2, wherein the inputs for the training sets include an attribute of a radiologist entity that provided the feedback score, and wherein the inputs to the deployed trained orchestration supervisor include an attribute of a radiologist entity to which the received patient medical image is to be forwarded with the at least one medical finding generated by the selected at least one analysis algorithm.

7. The computer program product of claim 2,
wherein the inputs for the training sets are selected from the group consisting of attributes of the training medical images, location of where the training medical images were generated, technology used to generate the training medical images, demographics of patients from which the training medical images were generated, attributes and identifiers of the analysis algorithms used to produce the classifications for which the feedback scores were provided, a location of a medical clinic that ordered the training medical image, and attributes of at least one radiologist entity that provided the feedback scores, and
wherein the inputs to the deployed trained orchestration supervisor are selected from the group consisting of attributes of the received patient medical image, location of where the received patient medical image was generated, technology used to generate the received patient medical image, demographics of a patient from which the received patient medical image was generated, attributes and identifiers of the analysis algorithms, a location of a medical clinic that ordered the received patient medical image for the patient, and attributes of a radiologist to which the received patient medical image and the at least one medical finding from the selected at least one analysis algorithm will be forwarded.

8. The computer program product of claim 2, wherein the feedback scores for the training medical findings from the analysis algorithm comprise feedback score vectors, wherein a feedback score vector for a training medical finding produced by an analysis algorithm for a training medical image includes an accuracy score indicating an accuracy of the training medical finding from the training medical image, a user experience score indicating a reviewing radiologist satisfaction with a user experience of the training medical finding from the training medical image, a performance score indicating a performance of the analysis algorithm producing the training medical finding, and a worth value indicating an extent to which a reviewing radiologist considers the analysis algorithm worth a cost, and
wherein the orchestration supervisor is trained to produce the feedback score vectors for the inputs from the training sets, and wherein algorithm scores for the analysis algorithms from the deployed trained orchestration supervisor comprise algorithm score vectors, wherein an algorithm score vector for an analysis algorithm of the analysis algorithms includes an accuracy score, a user experience score, a performance score, and a worth value.

9. The computer program product of claim 8, wherein the using the algorithm scores to select at least one analysis algorithm comprises:
aggregating, by an aggregation function, algorithm score vectors for the analysis algorithms into aggregation scores to rank the analysis algorithms to select the at least one analysis algorithm according to the rank of the analysis algorithms.

10. The computer program product of claim 8, wherein the using the algorithm scores to select at least one analysis algorithm comprises:
inputting the algorithm score vectors for the analysis algorithms into a learning-to-rank machine learning model to produce a ranking of the analysis algorithms to select the at least one analysis algorithm according to the ranking of the analysis algorithms.

11. The computer program product of claim 2, wherein the operations further comprise:
receiving indication that one of the analysis algorithms has been updated to an updated analysis algorithm;
generating training sets for the updated analysis algorithm including inputs comprising an attribute of the updated analysis algorithm, an attribute of training medical images, and the feedback scores for medical findings produced by the updated analysis algorithm; and
training the orchestration supervisor with the inputs from the training sets for the updated analysis algorithm to produce the feedback scores for the updated analysis algorithm based on the inputs.

12. A system for selecting one of a plurality of analysis algorithms to process a data set, comprising:
a processor;
an orchestration supervisor, implementing a machine learning algorithm; and
a computer readable storage medium having computer readable program code embodied therein that is executable by the processor to perform operations, the operations comprising:
receiving feedback scores for classifications produced by a plurality of analysis algorithms processing training data;
generating training sets including inputs comprising attributes of the analysis algorithms, attributes of the training data, and the feedback scores;
training an orchestration supervisor, implementing a machine learning algorithm, with the inputs from the training sets to produce the feedback scores;
deploying the trained orchestration supervisor to receive inputs comprising attributes of the analysis algorithms and attributes of received data to output algorithm scores for the analysis algorithms;
using the algorithm scores to select at least one analysis algorithm of the analysis algorithms; and
forwarding the received data to the selected at least one analysis algorithm to generate at least one classification.

13. The system of claim 12, wherein the training data comprises training medical images and the received data comprises a received patient medical image for a patient, wherein the classifications produced by the analysis algorithms comprise training medical findings for the training medical images and patient medical findings for the received patient medical image, and wherein the feedback scores are determined by at least one radiologist reviewing the training medical findings produced for the training medical images.

14. The system of claim 13,
wherein the inputs for the training sets are selected from the group consisting of attributes of the training medical images, location of where the training medical images were generated, technology used to generate the training medical images, demographics of patients from which the training medical images were generated, attributes and identifiers of the analysis algorithms used to produce the classifications for which the feedback scores were provided, a location of a medical clinic that ordered the training medical image, and attributes of at least one radiologist entity that provided the feedback scores, and
wherein the inputs to the deployed trained orchestration supervisor are selected from the group consisting of attributes of the received patient medical image, location of where the received patient medical image was generated, technology used to generate the received patient medical image, demographics of a patient from which the received patient medical image was generated, attributes and identifiers of the analysis algorithms, a location of a medical clinic that ordered the received patient medical image for the patient, and attributes of a radiologist to which the received patient medical image and the at least one medical finding from the selected at least one analysis algorithm will be forwarded.

15. The system of claim 13, wherein the feedback scores for the training medical findings from the analysis algorithm comprise feedback score vectors, wherein a feedback score vector for a training medical finding produced by an analysis algorithm for a training medical image includes an accuracy score indicating an accuracy of the training medical finding from the training medical image, a user experience score indicating a reviewing radiologist satisfaction with a user experience of the training medical finding from the training medical image, a performance score indicating a performance of the analysis algorithm producing the training medical finding, and a worth value indicating an extent to which a reviewing radiologist considers the analysis algorithm worth a cost, and
wherein the orchestration supervisor is trained to produce the feedback score vectors for the inputs from the training sets, and wherein algorithm scores for the analysis algorithms from the deployed trained orchestration supervisor comprise algorithm score vectors, wherein an algorithm score vector for an analysis algorithm of the analysis algorithms includes an accuracy score, a user experience score, a performance score, and a worth value.

16. The system of claim 13, wherein the operations further comprise:
receiving indication that one of the analysis algorithms has been updated to an updated analysis algorithm;
generating training sets for the updated analysis algorithm including inputs comprising an attribute of the updated analysis algorithm, an attribute of training medical images, and the feedback scores for medical findings produced by the updated analysis algorithm; and
training the orchestration supervisor with the inputs from the training sets for the updated analysis algorithm to produce the feedback scores for the updated analysis algorithm based on the inputs.

17. A computer implemented method for selecting one of a plurality of analysis algorithms to process a data set, comprising:
receiving feedback scores for classifications produced by a plurality of analysis algorithms processing training data;
generating training sets including inputs comprising attributes of the analysis algorithms, attributes of the training data, and the feedback scores;
training an orchestration supervisor, implementing a machine learning algorithm, with the inputs from the training sets to produce the feedback scores;
deploying the trained orchestration supervisor to receive inputs comprising attributes of the analysis algorithms and attributes of received data to output algorithm scores for the analysis algorithms;
using the algorithm scores to select at least one analysis algorithm of the analysis algorithms; and
forwarding the received data to the selected at least one analysis algorithm to generate at least one classification.

18. The method of claim 17, wherein the training data comprises training medical images and the received data comprises a received patient medical image for a patient, wherein the classifications produced by the analysis algorithms comprise training medical findings for the training medical images and patient medical findings for the received patient medical image, and wherein the feedback scores are determined by at least one radiologist reviewing the training medical findings produced for the training medical images.

19. The method of claim 18,
wherein the inputs for the training sets are selected from the group consisting of attributes of the training medical images, location of where the training medical images were generated, technology used to generate the training medical images, demographics of patients from which the training medical images were generated, attributes and identifiers of the analysis algorithms used to produce the classifications for which the feedback scores were provided, a location of a medical clinic that ordered the training medical image, and attributes of at least one radiologist entity that provided the feedback scores, and
wherein the inputs to the deployed trained orchestration supervisor are selected from the group consisting of attributes of the received patient medical image, location of where the received patient medical image was generated, technology used to generate the received patient medical image, demographics of a patient from which the received patient medical image was generated, attributes and identifiers of the analysis algorithms, a location of a medical clinic that ordered the received patient medical image for the patient, and attributes of a radiologist to which the received patient medical image and the at least one medical finding from the selected at least one analysis algorithm will be forwarded.

20. The method of claim 18, further comprising:
receiving indication that one of the analysis algorithms has been updated to an updated analysis algorithm;
generating training sets for the updated analysis algorithm including inputs comprising an attribute of the updated analysis algorithm, an attribute of training medical images, and the feedback scores for medical findings produced by the updated analysis algorithm; and
training the orchestration supervisor with the inputs from the training sets for the updated analysis algorithm to produce the feedback scores for the updated analysis algorithm based on the inputs.
